(19) United States
(12) Patent Application Publication
(10) Pub. No.: US 2020/0061371 A1
(43) Pub. Date: Feb. 27, 2020

(54) MEDICAL LEADS WITH SEGMENTED ELECTRODES AND METHODS OF FABRICATION THEREOF

(71) Applicant: ADVANCED NEUROMODULATION SYSTEMS, INC., Plano, TX (US)

(72) Inventor: Aaron Raines, Dallas, TX (US)

(21) Appl. No.: 16/108,838

(22) Filed: Aug. 22, 2018

(51) Int. Cl. A61N 1/05; B29C 45/00; A61N 1/36

(52) U.S. Cl. CPC A61N 1/0534; B29C 45/0055; B29C 45/14311; A61N 1/36062; A61N 1/0551

(57) ABSTRACT

A neurostimulation stimulation lead and method of fabrication are provided. First and second electrode sets include segmented electrodes joined with linking portions, bodies having a mandrel lumen, and a wire retention fixture. The second electrode set includes a wire pass through channel. The mandrel lumens, wire retention fixtures and wire pass through channel are monolithically formed in the corresponding bodies. The first and second wire filers are directly seated into the corresponding wire retention fixtures, such that the wire retention fixtures frictionally secure and retain the first and second wire filers. The first and second electrode sets are arranged in line with one another such that an intermediate portion of the first wire filer extends through the wire pass through channel in the second electrode set. The method removes the linking portions of the first and second electrode sets to unlink the segmented electrodes.

### BACKGROUND

Embodiments of the present disclosure generally relate to medical leads with segmented electrodes and fabrication thereof.

Deep brain stimulation (DBS) refers to the delivery of electrical pulses into one or several specific sites within the brain of a patient to treat various neurological disorders. For example, deep brain stimulation has been proposed as a clinical technique for treatment of chronic pain, essential tremor, Parkinson's disease (PD), dystonia, epilepsy, depression, obsessive-compulsive disorder, and other disorders.

A deep brain stimulation procedure typically involves first obtaining preoperative images of the patient's brain (e.g., using computer tomography (CT) or magnetic resonance imaging (MRI)). Using the preoperative images, the neurosurgeon can select a target region within the brain, an entry point on the patient's skull, and a desired trajectory between the entry point and the target region. In the operating room, the patient is immobilized and the patient's actual physical position is registered with a computer-controlled navigation system. The physician marks the entry point on the patient's skull and drills a burr hole at that location. Stereotactic instrumentation and trajectory guide devices are employed to control the trajectory and positioning of a lead during the surgical procedure in coordination with the navigation system.

Brain anatomy typically requires precise targeting of tissue for stimulation by deep brain stimulation systems. For example, deep brain stimulation for Parkinson's disease commonly targets tissue within or close to the subthalamic nucleus (STN). The STN is a relatively small structure with diverse functions. Stimulation of undesired portions of the STN or immediately surrounding tissue can result in undesired side effects. Mood and behavior dysregulation and other psychiatric effects have been reported from stimulation of the STN in Parkinson's patients.

To avoid undesired side effects in deep brain stimulation, neurologists often attempt to identify a particular electrode for stimulation that only stimulates the neural tissue associated with the symptoms of the underlying disorder while avoiding use of electrodes that stimulate other tissue. Also, neurologists may attempt to control the pulse amplitude, pulse width, and pulse frequency to limit the stimulation field to the desired tissue while avoiding other tissue.

As an improvement over conventional deep brain stimulation leads, leads with segmented electrodes have been proposed. Conventional deep brain stimulation leads include electrodes that fully circumscribe the lead body. Leads with segmented electrodes include, electrodes on the lead body that only span a limited angular range of the lead body.

Implementation of segmented electrodes are difficult due to the size of deep brain stimulation leads. Specifically, the outer diameter of deep brain stimulation leads can be approximately 0.06 inches or less. Fabricating electrodes to occupy a fraction of the outside diameter of the lead body and securing the electrodes to the lead body can be quite challenging.

An opportunity remains for lead designs that are smaller that conventional limits.

### SUMMARY

In accordance with embodiments herein a method is provided for fabricating a neurostimulation stimulation lead that comprises providing first and second electrode sets, each of which includes segmented electrodes joined with linking portions, the first and second electrode sets each including bodies having a mandrel lumen and a wire retention fixture, the body of the second electrode set including a wire pass through channel, the wire retention fixtures and wire pass through channel opening onto the corresponding mandrel lumens, wherein the mandrel lumens, wire retention fixtures and wire pass through channel are monolithically formed in the corresponding bodies. The method directly seats distal ends of first and second wire filers into the corresponding wire retention fixtures of the first and second electrode sets, respectively, such that the wire retention fixtures frictionally secure and retain the first and second wire filers. The method arranges the first and second electrode sets in line with one another such that an intermediate portion of the first wire filer extends through the wire pass through channel in the second electrode set. The method provides an outer tubing over the first and second electrode sets and wire filers extending from proximal ends of the first and second electrode sets and removes the linking portions of the first and second electrode sets to unlink the segmented electrodes.

Additionally or alternatively, the method bonds the first and second wire filers to the first and second electrode sets, respectively, wherein the seating operation retains the first and second filers directly engaged to, and seated in, the wire retention fixtures to form a non-hypo-tube connection there between during the bonding operation. Additionally or alternatively, the seating operation comprises introducing the distal ends of the first and second wire filers into the mandrel lumen and applying outward radial force to the distal ends of the first and second wire filers to snap and secure the distal ends into the corresponding wire retention fixtures. Additionally or alternatively, the method further comprises forming the mandrel lumen through the body of the first electrode set; forming at least two of the wire retention fixtures through the body of the first electrode set and opening onto the mandrel lumen; forming the wire pass through channel through the body of the first electrode set and opening onto the mandrel lumen; and forming an interstitial space through the body of the first electrode set and opening onto the wire pass through channel while leaving a linking portion of the body at a perimeter of the first electrode set to maintain first and second segmented electrodes in a linked state, the removing operation removing the linking portion to unlink the first and second segmented electrodes.

Additionally or alternatively, the method further comprises loading a non-metallic mandrel through the mandrel lumens in the first and second electrode sets, and through spacers separating the first and second electrode sets; and applying a heat shrinking operation that melts the outer tube, mandrel and spacers to fill the mandrel lumens, and to fill voids and interstitial spaces within and surrounding the electrode sets and wire filers, the outer tube, spacers and forming a lumen-less body.

Additionally or alternatively, the method the arranging operation further comprises loading a cord through the mandrel lumen of the second electrode set and thereafter, loading a proximal end of the first wire filer of the first electrode set through the wire pass-through channel in the second electrode set. Additionally or alternatively, after the removing operation, the first and second electrode sets have an outer diameter of less than 25 mils when in an unlinked state with the linking portions removed. Additionally or alternatively, an inner diameter of the wire retention fixtures generally corresponds to an outer diameter of the corresponding first and second wire filers, the wire retention fixtures opening onto the corresponding mandrel lumens at transition zones that have a width that is less than an outer diameter of the corresponding first and second wire filers.

In accordance with embodiments herein, a method is provided for fabricating a neurostimulation stimulation lead that comprises providing at least first and second electrode sets, each of which includes segmented electrodes joined with linking portions, the first and second electrode sets each including bodies having a mandrel lumen, bonding distal ends of first and second wire filers to the first and second electrode sets, respectively, and arranging the first and second electrode sets separated by spacers and in line with one another such that an intermediate portion of the first wire filer extends through the second linked electrode set. The method loads a non-metallic mandrel through the spacers and the mandrel lumens in the first and second electrode sets, provides an outer tubing over the first and second electrode sets and over the wire filers extending from proximal ends of the first and second electrode sets, applies a heat shrinking operation that melts the outer tube, mandrel and spacers to fill the mandrel lumens, voids and interstitial spaces within and surrounding the electrode sets and wire filers; and removes the linking portions of the first and second electrode sets to unlink the segmented electrodes.

Additionally or alternatively, the bodies of the first and second electrode sets each include a wire retention fixture, and the body of the second electrode set includes a wire pass through channel, the wire retention fixtures and wire pass through channel opening onto the corresponding mandrel lumens, wherein the mandrel lumens, wire retention fixtures and wire pass through channel are monolithically formed in the corresponding bodies. Additionally or alternatively, the method further comprises bonding the first and second wire filers to the first and second electrode sets, respectively, wherein the seating operation retains the first and second filers directly engaged and seated in the wire retention fixtures to form a non-hypo-tube connection there between during the bonding operation. Additionally or alternatively, the method further comprises directly seating distal ends of first and second wire filers into the corresponding wire retention fixtures of the first and second electrode sets, respectively, such that the wire retention fixtures frictionally secure and retain the first and second wire filers, wherein the seating operation comprises introducing the distal ends of the first and second wire filers into the mandrel lumen and applying outward radial force to the distal ends of the first and second wire filers to snap and secure the distal ends into the corresponding wire retention fixtures. Additionally or alternatively, the nonmetallic mandrel is formed of a polymer that melts to fuse with the outer tube and spacers to form a lumen-less body.

Additionally or alternatively, prior to the removing operation, the first and second electrode sets have an outer diameter of less than 40 mils when in a linked state with the linking portions joining the segmented electrodes, and wherein, after the removing operation, the first and second electrode sets have an outer diameter of less than 25 mils when in an unlinked state with the linking portions removed.

In accordance with embodiments herein, a neurostimulation stimulation lead is provide that comprises first and second electrode sets, each of which includes segmented electrodes, the first and second electrode sets including bodies having a mandrel lumen and a wire retention fixture, the body of the second electrode set including a wire pass through channel, the wire retention fixtures and wire pass through channel opening onto the corresponding mandrel lumens, wherein the mandrel lumens, wire retention fixtures and wire pass through channel are monolithically formed in the corresponding bodies; and wire filers having distal ends that are directly seating into the corresponding wire retention fixtures of the first and second electrode sets, respectively, such that the wire retention fixtures frictionally secure and retain the first and second wire filers. The first and second electrode sets are arranged in line with one another such that an intermediate portion of the first wire filer extends through the wire pass through channel in the second linked electrode set. The lead further comprises an outer tubing provided over the first and second electrode sets and over wire filers extending from proximal ends of the first and second electrode sets.

Additionally or alternatively, the lead further comprises bonds joining the first and second wire fliers to the first and second electrode sets, respectively, wherein the first and second filers are directly engaged and seated in the wire retention fixtures to form a non-hypo-tube connection there between during the bonding operation. Additionally or alternatively, an inner diameter of the wire retention fixtures generally corresponds to an outer diameter of the corresponding first and second wire filers, the wire retention fixtures opening onto the corresponding mandrel lumens at transition zones that have a width that is less than an outer diameter of the corresponding first and second wire filers. Additionally or alternatively, the lead further comprises a spacer between the first and second electrode sets and a non-metallic mandrel inserted through the spacers and the mandrel lumens hi the first and second electrode sets, the non-metallic mandrel, spacers and outer tube in a melted state fused with one another to fill the mandrel lumens, voids and interstitial spaces within and surrounding the electrode sets and wire filers. Additionally or alternatively, the first and second electrode sets have an outer diameter of less than 25 mils when in an unlinked state, the wire retention fixtures having an inner diameter that is substantially similar to an outer diameter of the wire filers, the wire pass through channels having an inner diameter larger than the outer diameter of the wire filers. Additionally or alternatively, the first electrode set includes at least three segmented electrodes of approximately equal arcuate size, each of the at least three segmented electrodes having a corresponding wire retention fixture formed on an interior surface thereof at the mandrel lumen, the wire retention fixtures in a central region of the corresponding segmented electrodes as measured along an arcuate path of an exterior surface of the corresponding segmented electrode.

### BRIEF DESCRIPTION OF THE DRAWINGS

FIG. 1 illustrates a side perspective view of a lead assembly formed in accordance with embodiments herein.

FIG. 2A illustrates a process for manufacturing the lead assembly hi accordance with embodiments herein.

FIG. 2B illustrates a process for manufacturing the lead assembly in accordance with embodiments herein,

FIG. 3A illustrates a front isometric view of a linked electrode set formed in accordance with embodiments herein.

FIG. 3B illustrates a front perspective view of the linked electrode set in accordance with embodiments herein.

FIG. 4A illustrates an end perspective view of an electrode assembly with wire filers at various stages of attachment in accordance with embodiments herein.

FIG. 4B illustrates an end perspective view of the linked electrode set to better illustrate features of the distal facing surface in accordance with embodiments herein.

FIG. 4C illustrates a fully assembled combination of two electrode subassemblies, with the polymer spacer removed and at a point in the assembly process prior to the centerless grinding operation in accordance with embodiments herein.

FIG. 4D illustrates the lead assembly after extruding the over tubing onto the assembled distal and proximal electrode subassemblies in accordance with embodiments herein.

FIG. 4E illustrates a perspective view of a fully assembled and heat shrunk assembled lead assembly with at least a portion of the outer tubing and spacers rendered partially transparent to show the internal components in accordance with embodiments herein.

FIG. 4F illustrates an end profile view of the unlinked electrode set after the centerless grinding operation in accordance with embodiments herein.

FIG. 5A illustrates an end profile view of a linked electrode set formed in accordance with embodiments herein.

FIG. 5B illustrates the electrode set in an unlinked state after a center-less grinding operation in accordance with embodiments herein.

FIG. 6A illustrates an end profile view of a linked electrode set formed in accordance with embodiments herein.

FIG. 6B illustrates an example in which combinations of segmented electrodes are maintained linked to one another while other combinations are unlinked in accordance with embodiments herein.

FIG. 7 illustrates a finished stimulation lead within a neurostimulation or other active medical device system in accordance with embodiments herein.

FIG. 8 illustrates a side perspective view of a distal portion of a lead assembly formed in accordance with alternative embodiments herein.

FIG. 9 illustrates a side perspective view of a distal portion of a lead assembly formed in accordance with alternative embodiments herein.

FIG. 10 illustrates a side perspective view of a distal portion of a lead assembly formed in accordance with alternative embodiments herein.

FIG. 11 illustrates a side perspective view of a distal portion of a lead assembly formed in accordance with alternative embodiments herein.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily ail referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The term “segmented electrode” is distinguishable from the term “ring electrode,” As used herein, the term “segmented electrode” refers to an electrode of a set of electrodes that are positioned at approximately the same longitudinal location along the longitudinal length of a lead and that are angularly positioned about the longitudinal axis so they do not overlap and are electrically isolated from one another. For example, at a given position longitudinally along the lead body, three electrodes can be provided with each electrode covering respective segments of less than 120* about the outer diameter of the lead body. By selecting between such electrodes, the electrical field generated by stimulation pulses can be more precisely controlled and, hence, stimulation of undesired tissue can be more easily avoided.

The term “linked electrode set” and “unlinked state” are used to refer to an electrode set prior to a centerless grinding operation or other operation wherein the segmented electrodes are electrically joined by linking portions arranged about a diameter of the electrode set. The linking portions and segmented electrodes are homogeneously formed with one another as a monolithic structure.

The term “unlinked electrode set” and “unlinked state” are used to refer to an electrode set after a centerless grinding operation or other operation that removes the linking portions to electrically isolate the segmented electrodes from one another.

The terms “directly engage”, “directly seat”, “non-hypo-tube connection” and variations thereof refer to a connection between wire filers and wire retention fixtures that are formed in a body of the electrode sets, without any hypo-tubes, secondary fixtures, or other structures.

The terms “non-lumen” and “lumen-less body” are used herein to refer to a body of the final lead assembly that does not include a lumen through the electrode sets and spacers. While an initial temporary mandrel lumen may be provided at certain stages of the assembly process, the temporary mandrel lumen is filled with a nonmetallic mandrel that is heated and melted to substantially fill and remove the initial temporary mandrel.

Embodiments herein generally relate to stimulation leads and methods for fabricating stimulation leads comprising multiple segmented electrodes. In one embodiment, the lead is adapted for deep brain stimulation (DBS), in other embodiments, the lead may be employed for any suitable therapy including spinal cord stimulation (SCS), peripheral nerve stimulation, peripheral nerve field stimulation, dorsal root or dorsal root ganglion stimulation, cortical stimulation, cardiac therapies, ablation therapies, etc.

In accordance with embodiments herein, a lead assembly is manufactured that includes multiple segmented electrodes that are directly bonded to wire filers and enclosed within a generally monolithic insulated body that is void of a mandrel lumen. Wire retention fixtures and other features described herein enable the lead assembly to be formed without the need or use of hypo-tubes, and thus allows the overall diameter of the lead assembly to be smaller than a conventional lead assembly utilizing hypo-tubes. For example, when a traditional lead assembly, formed utilizing hypo-tubes, may have a overall diameter of 50 mils, lead assemblies formed in accordance with embodiments herein may reduce the diameter of the overall lead assembly to 20 mils. Embodiments herein avoid the need to use hypo-tubes that are connected to the wire filer conductors and then welded to the electrodes, instead connecting the segmented electrodes directly to the wire filer conductions.

In addition, embodiments herein avoid the need for a metal mandrel and avoid the conventional use of retaining a mandrel lumen open after final assembly. Instead, embodiments herein insert a polymer mandrel into the mandrel lumen and melt the polymer mandrel to fill the mandrel lumen and voids in interstitial areas within the interior of the lead assembly to form a finalized non-lumen or lumen-less lead assembly. The melted polymer mandrel, when filling the finalized lead assembly, adds strength to the overall lead assembly.

Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference. Additionally or alternatively, the IMD may include one or more structural and/or functional aspects of the device(s) described in U.S. Pat. No. 9,216,285 “Leadless Implantable Medical Device Having Removable And Fixed Components” and U.S. Pat. No. 8.831,747 “Leadless Neurostimulation Device And Method Including The Same”, which are hereby incorporated by reference. Additionally or alternatively, the IMD may include one or more structural and/or functional aspects of the device(s) described in U.S. Pat. No. 8,391,980 “Method And System For Identifying A Potential Lead Failure In An implantable Medical Device” and U.S. Pat. No. 9,232,485 “System And Method For Selectively Communicating With An Implantable Medical Device”, which are hereby incorporated by reference.

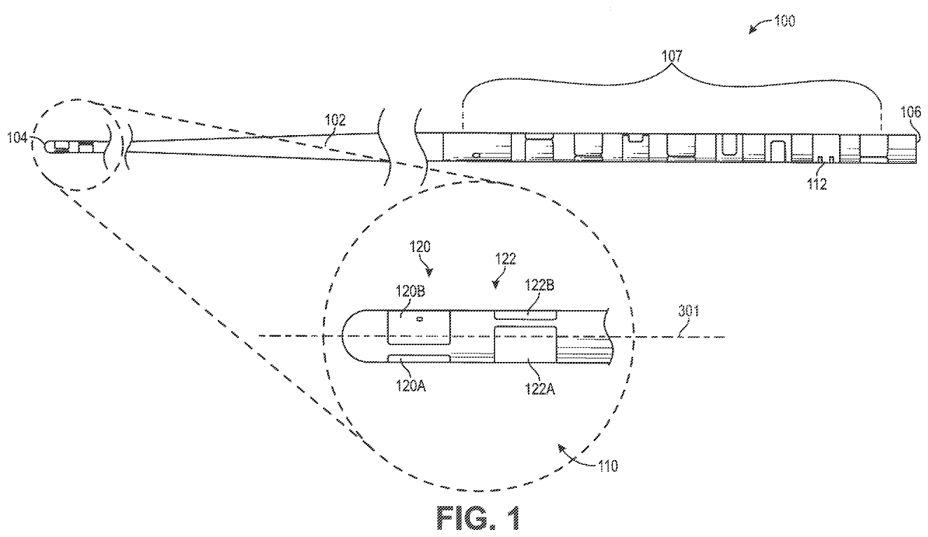

FIG. 1 Illustrates a side perspective view of a lead assembly 100 formed in accordance with embodiments herein. The lead assembly 100 includes a lead body 102 with a distal end 104 and a proximal end 106. The lead body 102 includes, at the distal end 104, a stimulation end component 110 that includes one or more segmented electrodes as shown in detail A. The stimulation end component 110 includes unlinked electrode sets 120, 122 formed on the lead body 102 about a longitudinal axis 301. The electrode set 120 has been separated into multiple segmented electrodes 120A, 1206, while the electrode set 122 has been separated into multiple segmented electrodes 122A, 122B.

The lead body 102 includes, at the proximal end 106, a terminal end component 107 that may be fabricated in various manners using suitable molding techniques. Terminal end component 107 may preferably comprise ring contacts for placement within the header of an implantable pulse generator (IPG). Terminal end component 107 may also comprise a non-active contact ring for use with a set screw and/or contact with an initial seal element within the header of the IPG. Terminal end component 107 preferably comprises a stylet guide and central lumen for the stylet. The terminal end component 107 includes a number of terminals 112 that corresponds to the number of segmented electrodes. The terminals 112 are configured to mate with corresponding contacts within a header of an implantable medical device when the proximal end 106 is inserted into the header. The terminals 112 are coupled to proximal ends of corresponding wire filers (not visible) that extend within and along the length of the lead body 102. Distal ends of the wire filers are coupled to corresponding segmented electrodes 120A, 1208, 122A, 1228 as described herein in more detail. Each terminal 112 and the corresponding segmented electrodes 120A, 1208, 122A, 122B form an electrically isolated circuit that may be utilized in connection with sensing and/or delivering stimulation. The terminals 112 and segmented electrodes 120A, 12013, 122A, 122B may be operated separately or in combinations.

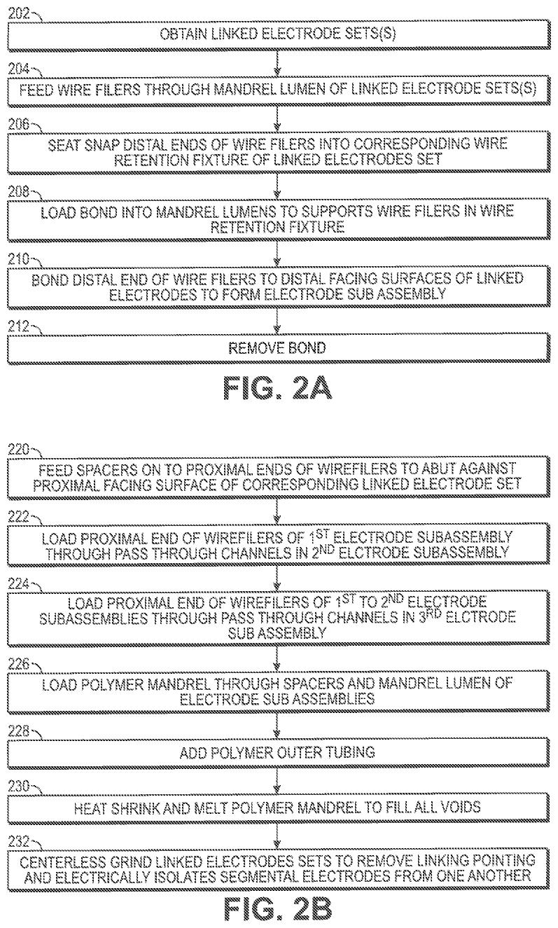

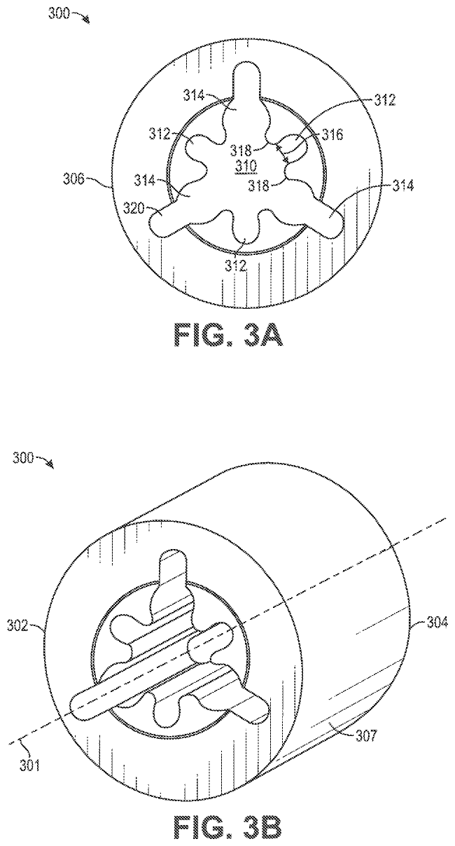

FIGS. 2A and 28 illustrate a process for manufacturing the lead assembly 100. At 202, one or more linked electrode sets are obtained. The obtaining operation may include manufacture and formation of the linked electrode sets and/or simply positioning one or more linked electrode sets in an electrode assembly tool. FIG. 3A illustrates a front isometric view of an electrode set 300 formed in accordance with an embodiment herein. FIG. 38 Illustrates a front perspective view of the linked electrode, set 300. The linked electrode set 300 includes a body formed in a generally disc shape about a longitudinal axis 301, where the body includes generally flat distal and proximal facing surfaces 302, 304 arranged parallel to one another and perpendicular to the longitudinal axis 301. The body of the linked electrode set 300 includes a generally circular side surface 306 extending along a circumference about the longitudinal axis 301. The linked electrode set 300 is in a linked state (hereafter referred to as a “linked electrode set”) that includes segmented electrodes joined by linking portions. The segmented electrodes and the linking portions are formed homogenous and monolithic with one another as the body of the electrode set 300.

A mandrel lumen 310 is formed in, and extends through, the body of the linked electrode, set 300. The mandrel lumen 310 is generally centered along the longitudinal axis 301. One or more wire retention fixtures 312 and wire pass-through channels 314 are also formed in, and extend through, the body of the linked electrode set 300. The mandrel lumen 310, wire retention fixtures 312 and wire pass-through channels 314 extend generally parallel to the longitudinal axis 301 to form openings between the distal and proximal facing surfaces 302, 304. The wire retention fixtures 312 are configured to retain wire filer& The wire retention fixtures 312 have a diameter that is generally the same as a diameter of the wire filers to be received therein. The wire pass-through channels 314 are also configured to receive wire filers, but have a diameter that is greater than the diameter of the wire retention fixtures 312 in order to allow the wire filers to freely move through the wire pass-through channels 314 in a longitudinal direction along the longitudinal axis 301.

In the example of FIGS. 3A, 3B, the linked electrode set 300 is formed with three wire retention fixtures 312 and three wire pass-through channels 314 that are evenly distributed about a perimeter of the mandrel lumen 310. The wire retention fixtures 312 and wire pass-through channels 314 open onto the mandrel lumen 310. The wire retention fixtures 312 are interleaved with the wire pass-through channels 314 in an alternating manner. Optionally, fewer or more wire retention fixtures 312 may be utilized and fewer or more wire pass-through channels 314 may be utilized. As a further option, more or fewer wire retention fixtures 312 may be provided relative to the number of wire pass-through channels 314, depending upon the overall configuration of the lead assembly 100.

The wire retention fixtures 312 and wire pass-through channels 314 are formed with a generally circular cross-section. The wire retention fixtures 312 open onto the mandrel lumen 310 at transition zones 316 that are located between adjacent ribs 318 extending along the body of the linked electrode set 300 and along the mandrel lumen 310. A transition zone 316 has a width between the adjacent ribs 318 that is smaller than a diameter of the wire retention fixture 312. The width of the transition zone 316 may be varied to achieve a desired retention force once a wire filer is snapped/pressed into the wire retention fixture 312.

The body of the linked electrode set 300 is also formed with interstitial spaces 320 that are aligned with, and open onto, the wire pass-through channels 314. The interstitial spaces 320 are distributed evenly about the longitudinal axis 301, and as explained herein, after a centerless grinding operation, the interstitial spaces 320 define separation points between segmented electrodes. In the example of FIGS. 3A and 3B, three interstitial spaces 320 are evenly distributed (e.g., at 120c′ angles with respect to one another about the longitudinal axis 301), such that three segmented electrodes of equal arcuate size will be formed following the centerless grinding operation. Optionally, the interstitial spaces 320 may be formed separate from, and not necessarily aligned with, the wire pass-through channels 314, such as when fewer or more wire pass-through channels 314 are desired relative to the number of interstitial spaces 320.

The dimensions of the linked electrode set 300 and dimensions of the features therein may be varied based on a desired overall dimension for the lead assembly 100, segmented electrodes, wire filers and the like. Embodiments herein are able to be repeatably and reliably manufactured with smaller dimensions as compared to conventional lead assemblies that utilize hypotubes. By way of example, the electrode sets 300 have an outer diameter of less than 40mils when in a linked state with the linking portions joining the segmented electrodes. More specifically, before the centerless grinding operation, the outer diameter of the linked electrode set 300 may be less than 40 mils (e,g., less than 0.04 inches), and as a further example between 10-40 mils (e.g., 0.01-0.04 inches), and as a further example between 25 and 35 mils, and as a further example approximately 30 mils. The outer diameter is measured to an outermost peripheral surface of the body of the electrode set 300. The wire retention fixtures having an inner diameter that is substantially similar to an outer diameter of the wire filers, and the wire pass through channels may have an inner diameter that is larger than the outer diameter of the wire filers. For example, an inner diameter of the wire retention fixtures may be approximately 3 mils, corresponding to an outer diameter of the wire filers, with the width of the transition zone 316 to be less than 3 mils (e.g., 2 3/4 mils, 2½ mils). The width of the transition zones 316 is less than an outer diameter of the wire filers to facilitate a snapping action and to retain the wire filers directly seated in the wire retention fixture 312. The width of the transition zones 316 may be adjusted to be sufficiently wide to allow the wire filers to be snapped/pressed into the wire retention fixture 312 when a sufficient outward radial force is applied to the wire filers, where the force is not so great as to deform or otherwise compromise the electrical or structural integrity of the wire filers. The width of the transition zones 316 may be also adjusted to be sufficiently narrow to retain the wire filers without any additional retention or fixture features. The wire retention fixtures 312, and transition zones 316 between ribs 318, are integral and monolithic parts of the body of the linked electrode set 300, thereby providing a self-fixturing functionality within the body of the linked electrode set 300 sufficient to retain the wire filers independent of, and without a need for, any other temporary or permanent structures. Among other things, the self-fixturing functionality provided by the wire retention fixtures 312 allows the lead assembly 100 to be formed without hypo-tubes as utilized in the configuration of U.S. Pat. No. 9,370.653, titled ‘Medical Leads with Segmented Electrodes and Methods of Fabrication Thereof,” the complete subject matter of which is incorporated herein by reference in its entirety.

The inner diameter of the wire pass-through channels is generally greater than a diameter of the wire filers by an amount sufficient to allow the wire filers to freely and slidably move through and rest therein. By way of example, the wire pass-through channels may have a diameter of approximately 5-10 mils, or another amount, provided that the diameter is greater than a diameter of the wire filers.

Returning to FIG. 2A, the linked electrode sets may be formed from various materials, such as platinum iridium. The linked electrode sets may be formed in various manners, such as utilizing a wire electrical discharge machine to cut the mandrel lumen, wire retention fixtures, wire pass-through channels and interstitial spaces into the body. Optionally, the linked electrode sets may be formed utilizing metal injection molding and other machining processes.

At 204, wire filers are formed and fed through the mandrel lumen of one or more linked electrode sets. For example, the wire fliers may include a set of one, three, five, seven or more individual wires wound in a helical manner and covered with insulation coating. Distal ends of the wire filers may have the insulation removed, such as through laser ablation. It is recognized that alternative configurations of wire filers may be utilized and may be formed in other manners. For example, the wire filers may he provided as a conductor cable, on which the ends are ablated to expose conductive material from insulative sheaths about the conductors. In one embodiment, one or more of the conductors are coated with a suitable dye material or other colorant to facilitate Identification of a specific channel in the finished stimulation tip component.

At 206, a seating/snapping operation applies an outward radial force to a distal end of the wire filer to secure/snap the distal end of the wire filer into the corresponding wire retention fixture of a corresponding linked electrode set. The operations at 204, 206 may be repeated in series or in parallel for multiple wire filers that are attached to a single linked electrode set. For example, when a linked electrode set includes three segmented electrodes, three wire filers may be fed into the mandrel lumen and radial forces applied in corresponding different radial directions to attach each wire filer to a corresponding segmented electrode. Optionally more than one wire filer may be attached to a single segmented electrode (e.g., when a single segmented electrode is formed with multiple wire retention fixtures). Optionally, not every segmented electrode may be connected to a wire filer (e.g., in configurations where a segmented electrode is to remain unused or otherwise coupled in common with another segmented electrode).

The operations at 204, 206 may be repeated in series or in parallel for multiple linked electrode sets. For example, a first group of two-four wire filers may be fed through the mandrel lumen of a first linked electrode set and radial forces applied to snap the distal ends into the corresponding wire retention fixtures. Separately or in parallel, a second group of two-four wire filers may be fed through the mandrel lumen of a second linked electrode set and radial forces applied to snap the distal ends into the corresponding wire retention fixtures.

At 208, a cord is loaded into the mandrel lumen to provide a secondary support for the wire filers, such as by preventing the wire filers from being inadvertently pulled out of or unsnapped from the wire retention fixtures due to inadvertent forces applied to a proximal end of the wire filers. Optionally, the operation at 208 may be omitted entirely with the wire retention fixtures operating alone to resist inadvertent detachment or un-snapping of the wire filers due to external forces applied thereto.

At 210, a bonding operation bonds the distal ends of the wire filers to the distal facing surface of the linked electrode set to form an electrode subassembly in which the wire filers are bonded to the linked electrode set. The bonding operation may be implemented in various manners. For example, the bonding operation may include various types of welding (e.g., laser welding, resistance welding). Additionally or alternatively, the bonding operation may include crimping the ribs 318 inward into the transition zone 316 (FIG. 3A). Additionally or alternatively, tabs may be formed along the ribs 318 that may be crimped into the transition zone 316. At 212, the cord is removed (if utilized at 208).

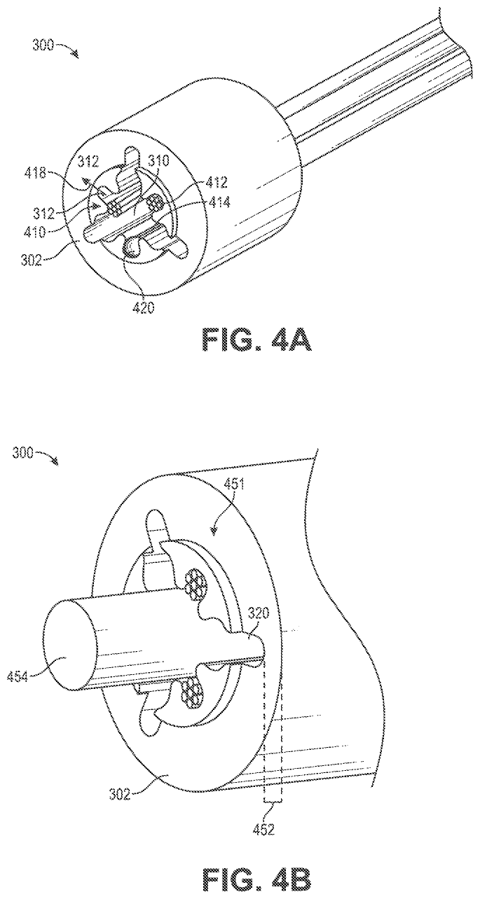

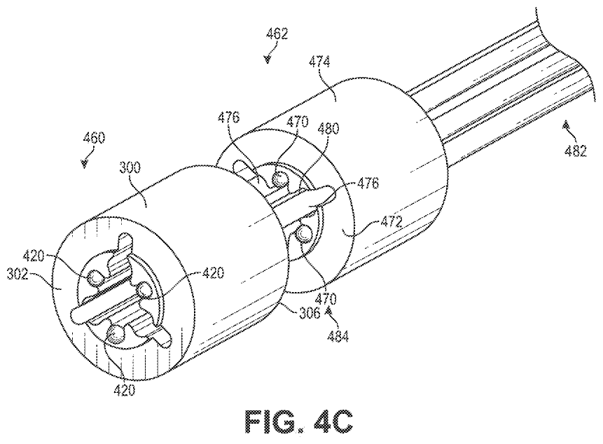

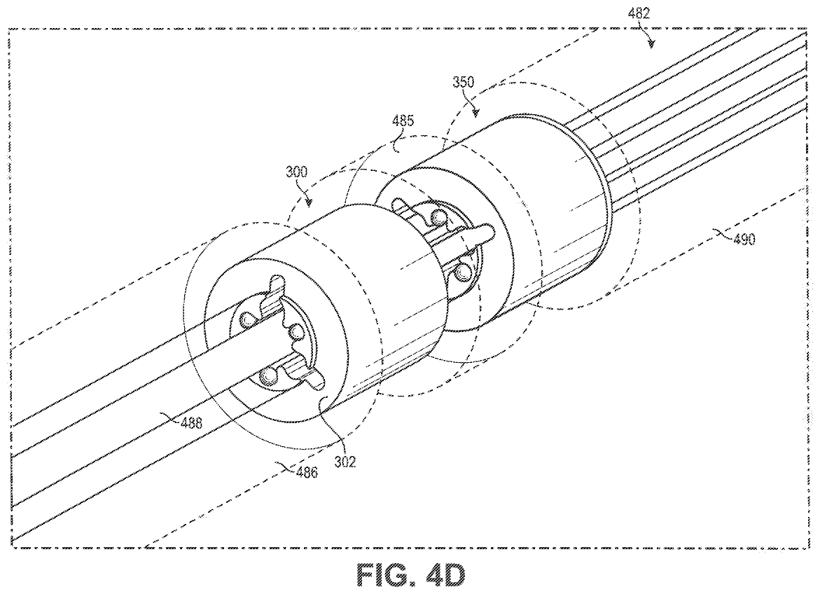

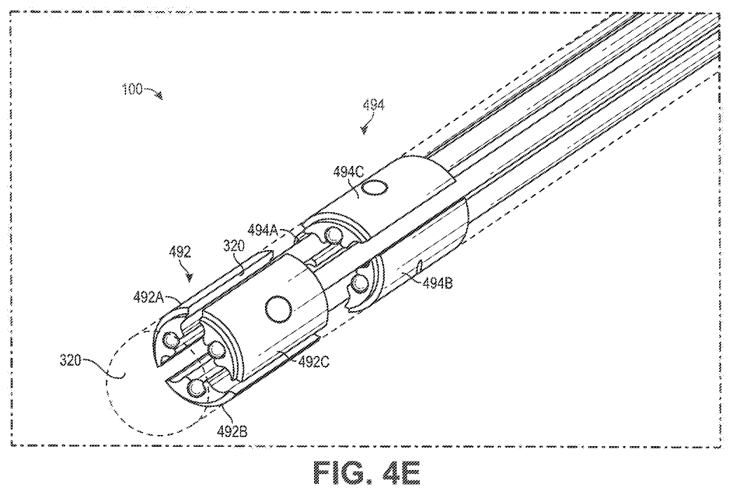

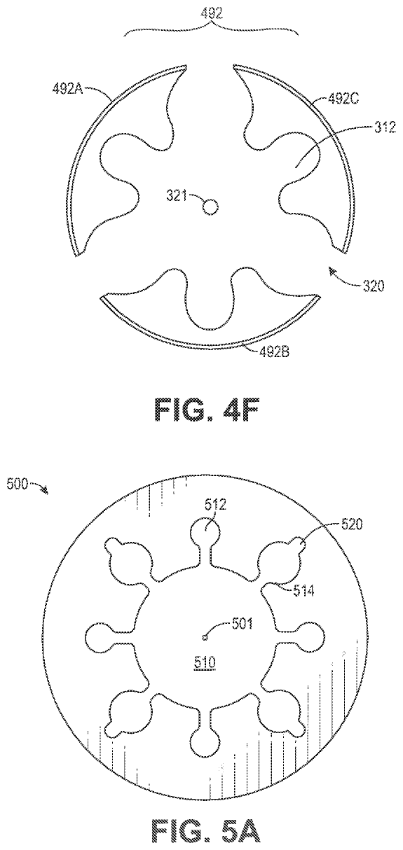

FIG. 4A illustrates an end perspective view of an electrode assembly with wire filers at various stages of attachment. FIG. 4A illustrates the linked electrode set 300 and wire filers 410, 412 and 414. The wire filers 410 are shown at the stage of assembly in which the wire filers have been inserted into the mandrel lumen 310, but not yet snapped into a corresponding wire retention fixture 312. At 206, a seating operation applies a radial force (corresponding to arrow 418) to the distal end of the wire filer 410 to force the wire filer 410 through the transition zone at an opening of the wire retention fixture 312. The outward radial force is applied until the wire filer is fully seated into the wire retention fixture, such as shown in connection with wire filer 412. When fully seated, the wire filer 412 is positioned to no longer interfere with the mandrel lumen 310 and generally passes through the transition zone 316 (FIG. 3A). Thereafter, the bonding operation at 210 bonds the distal end of the wire filers to the distal facing surface of the linked electrode set 300. For example, FIG. 4A illustrates the wire filer 414 to include a distal end that is bonded at weld 420 to a distal facing surface 302.

FIG. 4B illustrates an end perspective view of the linked electrode set 300 to better illustrate features of the distal facing surface 302. FIG. 4B further illustrates a cord 454 that may be inserted into the mandrel lumen 310 (FIG. 3A) at 208 to support the wire filers within the wire retention fixtures. The distal facing surface 302 includes one or more step-down regions 451 where the outer diameter is reduced. The step-down regions 451 may permit the linked electrode set 300 to be more securely integrated within the body of the stimulation end component 110 in the molding process. That is, the step-down regions 451 may be disposed below the outer surface of the insulation material after molding occurs. Also, an outer surface of the linked electrode set 300 may be bead blasted to increase the roughness of the surface of the body to improve bonding or adhesion to the insulation material. Also, the inner diameter (not shown) of linked electrode set 300 may be similar processed. Other techniques for application of abrasive materials to roughen the respective surfaces may be alternatively applied. The increase in surface roughness may further secure the integration of the metal components with the insulation material provided during the molding process.

FIG. 4B also illustrates the interstitial spaces 320 which comprise longitudinal grooves or cuts along the inner diameter of body to facilitate separation of the body into multiple segmented electrodes by a grinding process or other suitable processing. The reduced wall thickness (at 452) along such grooves permits separation during grinding operations as detailed in U.S. patent Ser. No. 12/873,838, filed Sep. 1, 2010 (published as U.S. Patent Pub. No. 2011/0047795) which is incorporated herein by reference.

Returning to FIG. 2A, following the operation at 212, flow moves to FIG. 2B. In FIG. 28, at 220, each electrode subassembly is further prepared by feeding spacers over proximal ends of the wire filers for the corresponding electrode subassembly. The spacers are advanced along the wire filers until located proximate to or abutting against the proximal facing surface of the corresponding linked electrode set. The spacers are formed of a polymer or other material that will bond to the electrode subassembly during a heating and shrinking operation described hereafter. The spacers are generally shaped as a ring with an outer perimeter having a diameter that is greater than the outer diameter of the linked electrode set. The spacers have a circular opening there through that may vary in size, but will generally be at least as large as the mandrel lumen.

At 222, first and second electrode subassemblies are assembled with one another by loading the proximal ends of the wire filers of a first electrode subassembly through the wire pass-through channels in a second electrode subassembly. At 224, the first and second electrode subassemblies are assembled with a third electrode subassembly by loading the proximal ends of the wire filers for the first and second electrode subassemblies through the wire pass-through channels in a third electrode subassembly. Optionally, the operation at 224 may be omitted entirely when only two electrode subassemblies are to be utilized. Optionally, the operation at 224 may be repeated multiple times when more than three electrode subassemblies are to be utilized.

The electrode subassembly configured to include the most distal segmented electrodes may represent a first electrode subassembly, with the wire filers thereof loaded through a second most distal electrode subassembly. It is recognized that the number of wire pass-through channels provided in the most proximal electrode subassembly may be greater than the number of wire pass-through channels in the most distal electrode subassembly. As a nonlimiting example configuration, assume two electrode subassemblies are to be utilized, with each electrode subassembly including three segmented electrodes. The distal electrode subassembly may not include any wire pass-through channels, whereas the proximal electrode subassembly would include at least three wire pass-through channels to account for the three separate wire filers associated with the three segmented electrodes on the distal electrode subassembly. As another nonlimiting example configuration, assume three electrode subassemblies are to be utilized, with each electrode subassembly including two segmented electrodes. The most distal electrode subassembly may not include any wire pass-through channels, whereas the intermediate electrode subassembly would include at least two wire pass-through channels to account for the two separate wire filers associated with the two segmented electrodes on the distal electrode subassembly. The most proximal electrode subassembly would include at least four wire pass-through channels to account for the separate wire filers associated with the two segmented electrodes on the distal electrode subassembly and the two segmented electrodes on the intermediate electrode subassembly.

FIG. 4C illustrates a fully assembled combination of two electrode subassemblies, with the polymer spacer removed and at a point in the assembly process prior to the centerless grinding operation. In FIG. 4C, a first/distal electrode subassembly 460 has been joined with a second/proximal electrode subassembly 462. In connection with the distal electrode subassembly 460, bonds 470 are visible between distal ends of three wire filers and the distal facing surface 302 of a distal linked electrode set 300. In connection with the proximal electrode subassembly 350, bonds 470 are visible between distal ends of two of the three wire filers and the distal facing surface 472 of a proximal linked electrode set 474. Also visible in FIG. 4C, wire filers 476 extend from the proximal end 306 of the distal linked electrode set 300 and pass-through corresponding wire pass-through channels 480 in the proximal linked electrode set 474. The wire filers 476 are avowed to freely slide within the wire pass-through channels 480 to form a cluster at 482 that includes six wire filers. While not illustrated, a polymer spacer is provided in the region denoted at 484 between the electrode subassemblies 460, 462. The polymer spacer maintains a predefined distance relation between the first and second electrode subassemblies 460, 462 along a longitudinal axis of the lead assembly.

Returning to FIG. 2B, once the electrode subassemblies are loaded with one another along with polymer spacers there between, flow advances to 226. At 226, the process loads a non-metallic mandrel through the spacers and through the mandrel lumen in the electrode subassemblies. By way of example, the nonmetallic mandrel may be formed of a polymer (e.g., nylon) or other heat-shrinkable material that will melt, fill the lumen and flow into any voids during a heat shrinking process described hereafter.

At 228, a polymer outer tubing is provided over the assembled combination of electrode subassemblies, as well as the polymer mandrel and the wire filers extending from the proximal end of the most proximal electrode subassembly. By way of example, the outer tubing may be formed of a Bionate™ polymer (thermoplastic polycarbonate urethane) or other insulative material. Optionally, the outer tubing may also be provided over the distal facing surface of the distal electrode subassembly. By way of example, the outer tubing may be added through an extrusion process in which the assembled electrode subassemblies and mandrel are pushed/pulled through an extrusion tool. Further, at 228, an outer cover is provided over the entire lead assembly.

FIG. 4D illustrates the lead assembly after extruding the over tubing onto the assembled distal and proximal electrode sets 300, 350. A spacer 485 is shown in the space between the distal and proximal electrode sets 300, 350. A distal outer tubing 486 is bonded to the distal facing surface 302 and surrounds a mandrel 488. A proximal outer tubing 490 surrounds and encloses the cluster 482 of wire filers extending from the proximal end of the proximal electrode subassembly 350. In addition, although not visible, an outer cover is provided over the entire lead assembly

Returning to FIG. 2B, at 230, a heat shrinking and melting operation is applied to the lead assembly. The heat shrinking operation melts the outer tubing and cover, as well as the mandrel, to fill substantially any and all voids and interstitial spaces within and surrounding the electrode subassemblies. Among other things, the polymer mandrel melts and flows into any gaps along the mandrel lumen, as well as a long the wire retention fixtures and wire pass-through channels to remove any air pockets or voids therein. The outer tubing similarly melts and bonds to an entire exterior surface of the electrode subassembly and flows into and fills the interstitial spaces and wire pass-through channels. The outer tubing, spacers and the mandrel melt and fuse with one another. By way of example, the outer tubing, spacers and mandrel may be formed of similar materials that fuse with one another to form a monolithic interface there between for a lumen-less body (e.g., no longer includes a lumen through the electrode sets and/or spacers).

In the example of FIG. 40, it is recognized that the spacer 485 and the distal and proximal outer tubing 486 and 490 have an outer diameter that is greater than an outer diameter of the linked electrode sets 300, 350, prior to the heat shrinking process. However, after the heat shrinking process, the outer tubing 486, 490 and spacer 485 shrink to have an outer diameter relatively similar to, or slightly less than, the outer diameter of the linked electrode sets 300, 350.

Returning to FIG. 2B, the process moves from 230, to 232. At 232, a centerless grinding operation is performed upon the linked electrode sets to remove the linking portions and electrically isolate each of the segmented electrodes from one another. By way of example, the centerless grinding operation may be performed in connection with the operations described in U.S. Patent Pub, No. 2011/0047795. The centerless grinding operation removes an outer portion of the linking electrode sets, thereby reducing an outer diameter for the final unlinked electrode sets. For example, the centerless grinding operation may remove 5-15 mils from the diameter, and as a further example, remove 10 mils, such that the outer diameter of the unlinked electrode sets may be 15-25 mils, and as a further example, approximately 20 mils. As a further example, the unlinked electrode sets may have an outer diameter of less than 25 mils when in the unlinked state with the linking portions removed.

FIG. 4E illustrates a perspective view of a fully assembled and heat shrunk assembled lead assembly 100 with at least a portion of the outer tubing and spacers rendered partially transparent to show the internal components. While not shown, the non-metallic mandrel, spacers and outer tube are in a melted state fused with one another to fill the mandrel lumens, voids and interstitial spaces within and surrounding the electrode sets 492, 494 and wire filers. In FIG. 4E, the centerless grinding operation has converted the distal and proximal linked electrode sets 300, 350 (FIG. 4D) into distal and proximal unlinked electrode sets 492, 494, each of which comprises a group of three individual segmented electrodes 492A. 492B, 492C, 494A, 494B and 494C. The segmented electrodes 492A, 492B, 492C, 494A, 494B and 494C are electrically separated from one another by interstitial spaces 320, such that each of the individual segmented electrodes 492A. 492B, 492C, 494A. 494B and 494C can separately be controlled to perform sensing and deliver stimulus through the corresponding wire filer bonded thereto.

FIG. 4F illustrates an end profile view of the unlinked electrode set 492 after the centerless grinding operation. The segmented electrodes 492A, 492B, 492C are separated at interstitial spaces 320. The wire retention fixtures 312 are formed to be generally in a central region of the corresponding segmented electrodes 492A, 492B, 492C as measured along the arcuate path of the exterior surface of the corresponding segmented electrode 492A, 492B, 492C from a center point 321 corresponding to the longitudinal axis 301 (FIG. 3B) of the lead body. Optionally, the wire retention fixtures 312 may be located at alternative positions along the interior surface of the corresponding segmented electrodes. Additionally or alternatively, more than one wire retention fixture may be formed in a single segmented electrode. Additionally or alternatively, more than one wire pass through channel may be formed in a single segmented electrode.

As noted herein, embodiments may be constructed with more or fewer segmented electrodes in each electrode set. For example each electrode set may include two segmented electrodes that are separated at interstitial spaces arranged at approximately 108° apart from one another. Alternatively, one or more electrode sets may include four, five or more segmented electrodes that are separated at interstitial spaces arranged at approximately 90° apart, 72° apart and the like.

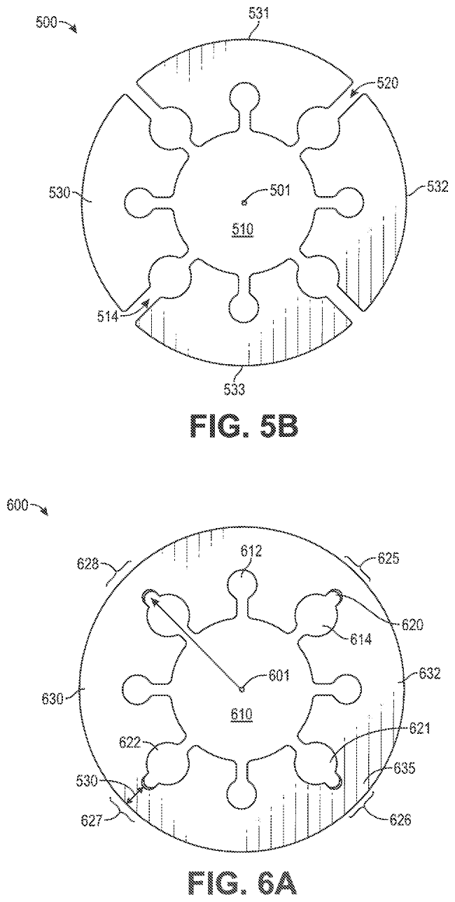

FIG. 5A illustrates an end profile view of a linked electrode set 500 formed in accordance with an alternative embodiment. The linked electrode set 500 is configured to be separated into four separate segmented electrodes. The linked electrode set 500 includes a body formed in a generally disc shape with flat distal and proximal facing surfaces (similar to the arrangement of FIGS. 3A and 3B). A mandrel lumen 510 is formed in, and extends through, the body of the linked electrode set 500. The mandrel 510 is generally centered along the longitudinal axis 501 (noted as a dot coming out of the drawings sheet).

Four wire retention fixtures 512 and four wire pass-through channels 514 are formed in, and extend through, the body of the linked electrode set 500. The wire retention fixtures 512 are sized to frictionally retain wire filers one seated therein. The wire retention fixtures 512 have a diameter that is generally the same as a diameter of the wire filers to be received therein. The wire pass-through channels 514 are configured to freely receive wire filers, and thus have a diameter that is sufficiently greater than the diameter of the wire retention fixtures 512 in order to allow the wire filers to freely slide along the wire pass-through channels 514 in the longitudinal direction corresponding to the longitudinal axis 501.

In the example of FIG. 5A, the four wire retention fixtures 512 and four wire pass-through channels 514 are evenly distributed about a perimeter of the mandrel lumen 510. The wire retention fixtures 612 and wire pass-through channels 514 open onto the mandrel lumen 510. The wire retention fixtures 512 are interleaved with the wire pass-through channels 514 in an alternating manner. Interstitial spaces 520 are distributed evenly about the longitudinal axis 501 and open onto the wire pass-through channels 514.

FIG. 5B illustrates the electrode set 500 in an unlinked state after a center-less grinding operation. The interstitial spaces 520 define separation points between four segmented electrodes 530-533. In the example of FIGS. 5A and 5B, four interstitial spaces 520 are evenly distributed (e.g., at 90° angles with respect to one another about the longitudinal axis 501), such that four segmented electrodes 630-533 of equal size will be formed following the centerless grinding operation.

Additionally or alternatively, the stimulation end component 110 may also include a radio-opaque marker to permit the orientation of the lead to be determined post-implant using suitable medical imaging. Stimulation end component 110 preferably includes a plurality of segmented electrodes, alone or in combination with one or more ring electrodes. For example, a ring electrode may be provided distal to and/or proximal to the segmented electrodes, although any suitable electrode configuration may be selected. The foregoing examples illustrated two rows of three segmented electrodes. Optionally, another possible electrode configuration includes two rows of four segmented electrodes. Another possible electrode configuration includes four rows of two segmented electrodes.

In the foregoing examples, the segmented electrodes are shown to be of substantially similar size when the linking portions are removed. Additionally or alternatively, one or more segmented electrodes may be formed to be larger than other segmented electrodes within a common electrode set. For example, and electrode set may include three segmented electrodes where a first segmented electrode is substantially the same size as the second and third segmented electrodes combined. Optionally, and electrode set may be formed with four segmented electrodes, wherein a first pair of opposed segmented electrodes are substantially larger than a second set of opposed segmented electrodes.

In the foregoing examples, the linking portions at each of the interstitial spaces have a substantially common thickness, such that during a centerless grinding operation, all of the linking portions are removed at the same time (e.g., when the outer 10 mils of the diameter of the electrode set are round away). Optionally, interstitial spaces may be formed with different radial depths, such that the linking portions have correspondingly different thicknesses. For example, a first interstitial space may be cut a first radial distance from the center of the electrode set, while a second interstitial space is cut a second radial distance from the center of the electrode set, thereby forming corresponding first and second linking portions having first and second different thicknesses. By providing different thicknesses to the linking portions, embodiments herein afford the ability to unlink a desired combination of segmented electrodes, without unlinking all segmented electrodes.

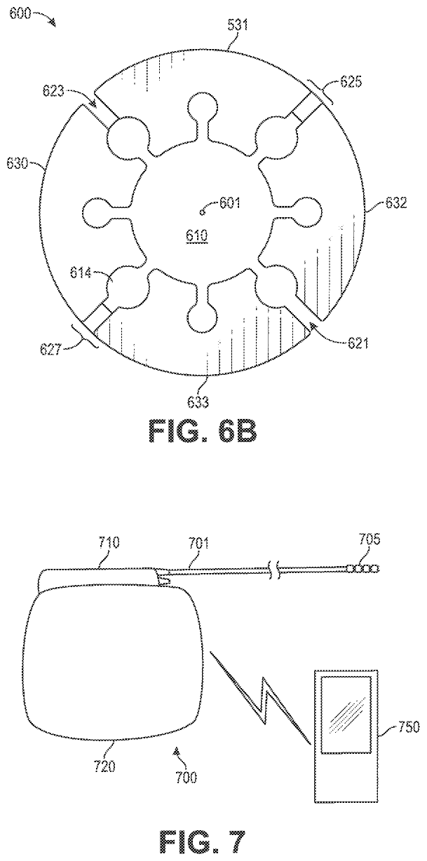

FIG. 6A illustrates an end profile view of a linked electrode set 600 formed in accordance with an alternative embodiment. The linked electrode set 600 is configured to be separated into two or four separate segmented electrodes depending upon adept to which the centerless grinding operation is performed. The linked electrode set 600 includes a body formed in a generally disc shape with flat distal and proximal facing surfaces (similar to the arrangement of FIGS. 3A and 3B). A mandrel lumen 610 is formed in, and extends through, the body of the linked electrode set 600. The mandrel 610 is generally centered along the longitudinal axis 601 (noted as a dot coming out of the drawings sheet).

Four wire retention fixtures 612 and four wire, pass-through channels 614 are formed in, and extend through, the body of the linked electrode set 600. The four wire retention fixtures 612 and four wire pass-through channels 614 are evenly distributed about a perimeter of the mandrel lumen 610. The wire retention fixtures 612 and wire pass-through channels 614 open onto the mandrel lumen 610. The wire retention fixtures 612 are interleaved with the wire pass-through channels 614 in an alternating manner.

Interstitial spaces 620-623 are distributed evenly about the longitudinal axis 601 and open onto the wire pass-through channels 614. The interstitial spaces 620-623 correspond to linking portions 625-628, respectively, which in turn separate segmented electrodes 630-633. The interstitial spaces 620-623 are cut to different radial depths 624, in order to form linking portions 625-628 that have different thicknesses 629. For example, the interstitial spaces 620 and 622 are arranged substantially 180° apart along and extend in opposite directions along a generally common diameter. The interstitial spaces 620 and 622 are cut to a common depth from the center point 602, thereby forming linking portions 625, 627 that have a common thickness 629. The interstitial spaces 621 and 623 are arranged substantially 180C apart along and extend in opposite directions along a generally common diameter. The interstitial spaces 621 and 623 are generally aligned along an axis orthogonal to the axis intersecting interstitial spaces 620 and 622. The interstitial spaces 621 and 623 are cut to a common depth from the center point 602, thereby forming linking portions 626, 628 that have a common thickness 635. The thickness 635 of the linking portions 626, 628 is less than the thickness 629 of the linking portions 625, 627, thereby affording a selective on linking operation based on the depth to which the electrode set is centerless ground.

By way of example, the linking portions 625, 627 may be formed to have a thickness of 12 mils, while the linking portions 626, 628 may be formed to have a lesser thickness of 8 mils. When it is desired to unlink certain combinations of segmented electrodes, but retain the link between certain other combinations of segmented electrodes, the centerless grinding operation may remove the more narrow linking portions while retaining the thicker linking portions.

FIG. 6B illustrates an example in which combinations of segmented electrodes are maintained linked to one another while other combinations are unlinked in accordance with embodiments herein. In the example of FIG. 66, it was desirable to retain the linking portion 625 between the pair of segmented electrodes 631 and 632, and to retain the linking portion 627 between the pair of segmented electrodes 630, 633. At the same time, it was desirable to remove the linking portion 626 between segmented electrodes 632, 633 and to remove the linking portion 628 between the segmented electrodes 631, 630. To do so, the centerless grinding operation removed 8 mils of the peripheral layer of the electrode set 600, thereby removing linking portions 626, 628, while leaving at least four mils of the linking portions 625, 627, thereby forming one dual segmented electrode 631, 632 and a second dual segmented electrode 630, 633.

Alternatively, when it is desirable to utilize for separate segmented electrodes, the centerless grinding operation may remove all 12 mils of the outer layer to remove all four linking portions 625-628.

FIG. 7 depicts a finished stimulation lead within a neurostimulation or other active medical device system according to embodiments herein. Neurostimulation system 700 includes pulse generator 720 and one or more stimulation leads 701. Examples of commercially available pulse generator include the EON™, EON MIMI™, LIBRA™, and BRIO™ pulse generators available from St. Jude Medical, Inc. Other active medical devices could be employed such as pacemakers, implantable cardioverter defibrillator, gastric stimulators, functional motor stimulators, etc. Pulse generator 720 is typically implemented using a metallic housing that encloses circuitry for generating the electrical pulses for application to neural tissue of the patient. Control circuitry, communication circuitry, and a rechargeable battery (not shown) are also typically included within pulse generator 720. Pulse generator 720 is usually implanted within a subcutaneous pocket created under the skin by a physician.

As fabricated according to techniques described herein, lead 701 is electrically coupled to the circuitry within pulse generator 720 using header 710. Lead 701 includes terminals (not shown) that are adapted to electrically connect with electrical connectors (e.g., “Bal-Seal” connectors which are commercially available and widely known) disposed within header 710. The terminals are electrically coupled to conductors (wire filers) within the lead body of lead 701: The conductors conduct pulses from the proximal end to the distal end of lead 701. The conductors are also electrically coupled to electrodes 705 to apply the pulses to tissue of the patient. Lead 701 can be utilized for any suitable stimulation therapy. For example, the distal end of lead 701 may be implanted within a deep brain location or a cortical location for stimulation of brain tissue. The distal end of lead 701 may be implanted in a subcutaneous location for stimulation of a peripheral nerve or peripheral nerve fibers. Alternatively, the distal end of lead 701 positioned within the epidural space of a patient. Although some embodiments are adapted for stimulation of neural tissue of the patient, other embodiments may stimulate any suitable tissue of a patient (such as cardiac tissue). An “extension” lead (not shown) may be utilized as an intermediate connector if deemed appropriate by the physician.

Electrodes 705 include multiple segmented electrodes. The use of segmented electrodes permits the clinician to more precisely control the electrical field generated by the stimulation pulses and, hence, to more precisely control the stimulation effect in surrounding tissue. Electrodes 705 may also include one or more ring electrodes and/or a tip electrode. Any of the electrode assemblies and segmented electrodes discussed herein can be used for the fabrication of electrodes 705. Electrodes 705 may be utilized to electrically stimulate any suitable tissue within the body including, but not limited to, brain tissue, tissue of the spinal cord, peripheral nerves or peripheral nerve fibers, digestive tissue, cardiac tissue, etc. Electrodes 705 may also be additionally or alternatively utilized to sense electrical potentials in any suitable tissue within a patient's body.

Pulse generator 720 preferably wirelessly communicates with programmer device 750. Programmer device 750 enables a clinician to control the pulse generating operations of pulse generator 720. The clinician can select electrode combinations, pulse amplitude, pulse width, frequency parameters, and/or the like using the user interface of programmer device 750. The parameters can be defined in terms of “stir sets,” “stimulation programs,” (which are known in the art) or any other suitable format. Programmer device 750 responds by communicating the parameters to pulse generator 720 and pulse generator 720 modifies its operations to generate stimulation pulses according to the communicated parameters.

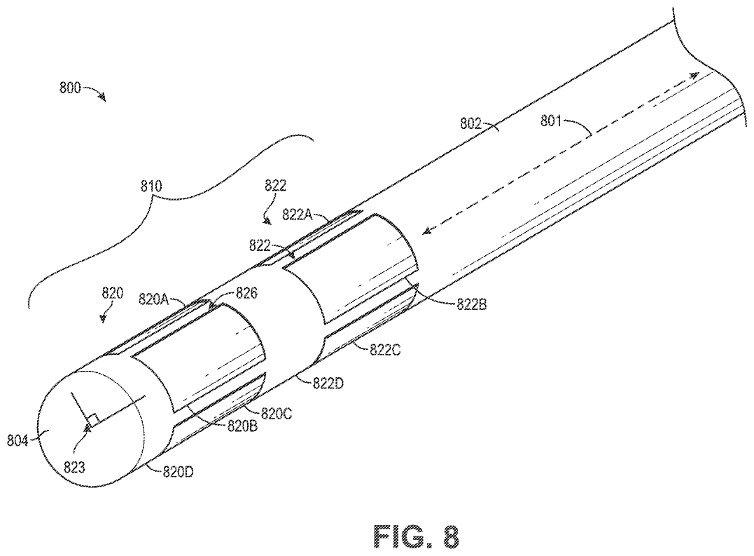

FIG. 8 illustrates a side perspective view of a distal portion of a lead assembly 800 formed in accordance with alternative embodiments herein. The lead assembly 800 includes a lead body 802 with a distal end 804. The lead body 802 includes, at the distal end 804, a stimulation end component 810 that includes first and second rows of unlinked electrode sets 820 and 822 that are spaced apart from one another along the longitudinal axis 801 of the lead body. The first row of the unlinked electrode set 820 is located adjacent the distal end 804. The second row of the unlinked electrode set 822 is located further from the distal end 804 at an intermediate point along the stimulation end component 810.

The electrode sets 820 and 822 each comprise a group of four individual segmented electrodes 820A-820D and 822A-822D, respectively (of which three are visible). The electrode set 820 has been separated into quarter-sector segmented electrodes 820A-820D that are arranged along arcuate outer sections of the lead body 802 and are generally centered along radius that are oriented at 90° with respect to one another as indicated by arc 823. Similarly, the electrode set 822 has been separated into quarter-sector segmented electrodes 822A 822D that are arranged along arcuate outer sections of the lead body and are generally centered along radius that are oriented at 90° with respect to one another as indicated by the same arc 823.

The first and second rows of electrode sets 820 and 822 are circumferentially aligned with one another. The term “circumferentially aligned” refers to a configuration in which the individual segmented electrodes 820A 820D, 822A-822D are positioned at common radial angles about the longitudinal axis 801. When in a circumferential alignment, the individual segmented electrodes 820A 820D from the distal electrode set 820 are aligned along the longitudinal axis 801 with the individual segmented electrodes 822A-822D to provide common continuous gaps 826, 827 therebetween. More specifically, the gap 826 is provided between segmented electrodes 820A, 820B in the electrode set 820, while the gap 827 is provided between segmented electrodes 822A, 8226 in the electrode set 822. The segmented electrodes 820A and 822A aligned with one another, as do the segmented electrodes 8206, 8226, and gaps 826, 827. The foregoing pattern is maintained about a circumference of the lead body 802.

Optionally, additional rows of unlinked electrode sets may be provided at more proximal positions along the lead body 802.

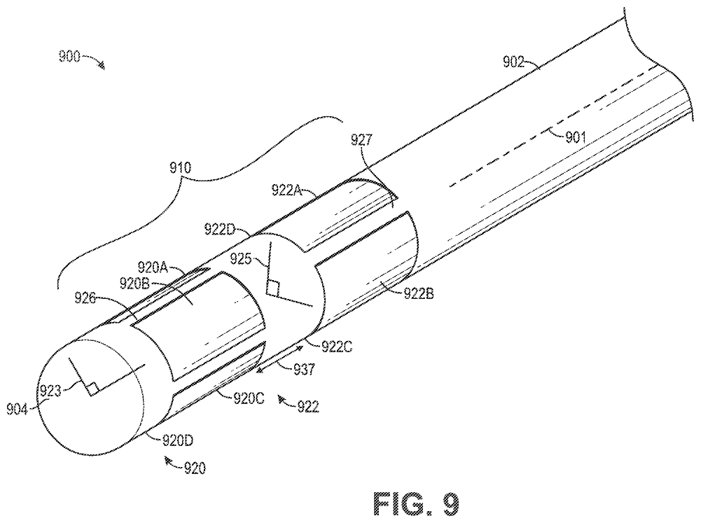

FIG. 9 illustrates a side perspective view of a distal portion of a lead assembly 900 formed in accordance with alternative embodiments herein. The lead assembly 900 includes a lead body 902 with a distal end 904. The lead body 902 includes, at the distal end 904, a stimulation end component 910 that Includes first and second rows of unlinked electrode sets 920 and 922 that are spaced apart from one another along the longitudinal axis 901 of the lead body by a longitudinal spacing 937. The first row of the unlinked electrode set 920 is located adjacent the distal end 904, while the second row of the unlinked electrode set 922 is located further from the distal end 904 at an intermediate point along the stimulation end component 910.

The electrode sets 920 and 922 each comprise a group of four individual segmented electrodes 920A-9200 and 922A-922D, respectively. The electrode set 920 has been separated into quarter-sector segmented electrodes 920A-920D that are arranged along arcuate quarter outer sections of the lead body 902 and are generally centered along radius that are oriented at 90° with respect to one another as indicated by arc 923. Similarly, the electrode set 922 has been separated into quarter-sector segmented electrodes 922A-922D that are arranged along arcuate outer sections of the lead body and are generally centered along radius that are oriented at 90° with respect to one another as indicated by the separate arc 925.

The first and second rows of electrode sets 920 and 922 are rotated with respect to one another about the circumference to be 45° out of rotational alignment or phase with one another. The term “out of rotational alignment” refers to a configuration in which the individual segmented electrodes 920A-920D, 922A-922D are positioned at different radial angles about the longitudinal axis 901, such as denoted by the arcs 923 and 925. When out of rotational alignment, the individual segmented electrodes 920A-920D from the distal electrode set 920 overlap, in the longitudinal direction, a gap between adjacent segmented electrodes 922A-922D to provide intermittent gaps 926, 927 therebetween. More specifically, the gap 926, between segmented electrodes 920A, 920B in the electrode set 920, is aligned with the segmented electrode 922A, the gap 927, between segmented electrodes 922A, 922B, is aligned with the segmented electrode 920B. The foregoing pattern is maintained about a circumference of the lead body 902.

Optionally, additional rows of unlinked electrode sets may be provided at more proximal positions along the lead body 902. Optionally, the rows of unlinked electrode sets may be rotated at different angles with respect to one another, other than at a 45° angle as denoted between arcs 923 and 925. Optionally, the rows of unlinked electrode sets may include different numbers of segmented electrodes.

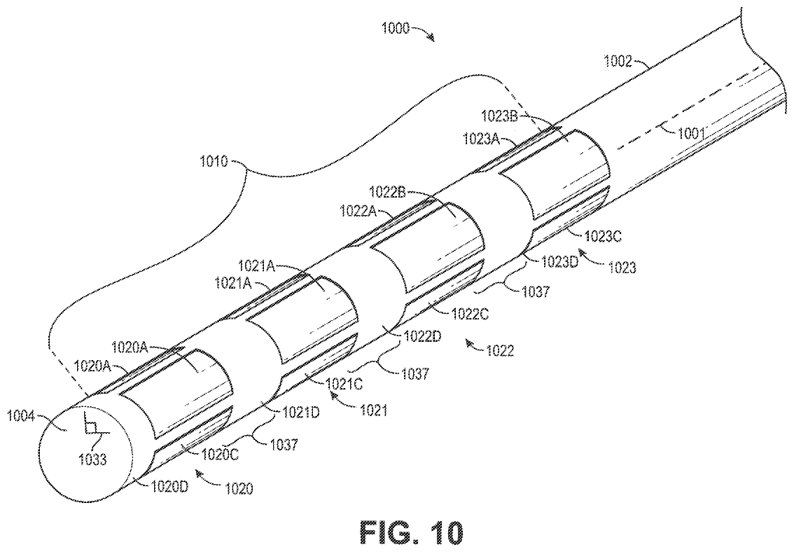

FIG. 10 illustrates a side perspective view of a distal portion of a lead assembly 1000 formed in accordance with alternative embodiments herein. The lead assembly 1000 includes a lead body 1002 with a distal end 1004. A stimulation end component 1010 includes four rows of unlinked electrode sets 1020-1023 that are spaced apart from one another along the longitudinal axis 1001 of the lead body by longitudinal spaces 1037 that may be the same or different. The first row of the unlinked electrode set 1020 is located adjacent the distal end 1004, while the second, third and fourth rows of the unlinked electrode sets 1021-1023 are located progressively further from the distal end 1004 along the stimulation end component 1010.

The electrode sets 1020-1023 each comprise a group of four individual segmented electrodes 1020A-10200, 1021A-1021D, 1022A-1022D and 1023A-1023D, respectively. The electrode sets 1020-1023 have been separated into quarter-sector segmented electrodes that are arranged along arcuate quarter outer sections of the lead body 1002 and are generally centered along radius that are oriented at 90° with respect to one another as indicated by arc 1033. The four rows of electrode sets 1020-1023 are circumferentially aligned with one another, such that the individual segmented electrodes 1020A-1020D, 1021A-1021D, 1022A-1022D and 1023A-1023D are positioned at common radial angles about the longitudinal axis 1001.

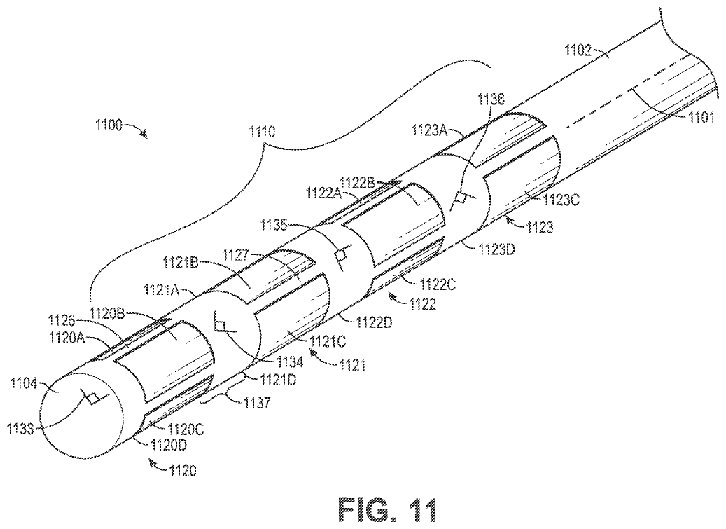

FIG. 11 illustrates a side perspective view of a distal portion of a lead assembly 1100 formed in accordance with alternative embodiments herein. The lead assembly 1100 includes a lead body 1102 with a distal end 1104. A stimulation end component 1110 includes four rows of unlinked electrode sets 1120-1123 that are spaced apart from one another along the longitudinal axis 1101 of the lead body by longitudinal spaces 1137 that may be the same or different. The first row of the unlinked electrode set 1120 is located adjacent the distal end 1104, while the second, third and fourth rows of the unlinked electrode sets 1121-1123 are located progressively further from the distal end 1104 along the stimulation end component 1110.

The electrode sets 1120-1123 each comprise a group of four individual segmented electrodes 1120A-11200, 1121A-1121D, 1122A-1122D and 1123A-1123D, respectively. The electrode sets 11201123 have been separated into quarter-sector segmented electrodes that are arranged along arcuate quarter outer sections of the lead body 1102 and are generally centered along radius that are oriented at 90° with respect to one another as indicated by arcs 1133-1136. The four rows of electrode sets 1120-1123 rotated with respect to one another about the circumference to be 45° out of rotational alignment or phase with one another, to provide a configuration in which the individual segmented electrodes 1120A-1120D, 1122A-1122D are positioned at different radial angles about the longitudinal axis 1101, such as denoted by the arcs 1133-1136. When out of rotational alignment, the individual segmented electrodes 1120A-1120D from the distal electrode set 1120 overlap, in the longitudinal direction, a gap between adjacent segmented electrodes 1122A-1122D to provide intermittent gaps 1126, 1127 therebetween. The foregoing pattern is maintained about a circumference of the lead body 1102.

Optionally, additional rows of unlinked electrode sets may be provided at more proximal positions along the lead body 1102. Optionally, the rows of unlinked electrode sets may be rotated at different angles with respect to one another, other than at a 45° angle as denoted between arcs 1133-1136. Optionally, the rows of unlinked electrode sets may include different numbers of segmented electrodes.

### Closing Statements

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass—the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims ar entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A method for fabricating a neurostimulation stimulation lead comprising:
providing first and second electrode sets, each of which includes segmented electrodes joined with linking portions, the first and second electrode sets each including bodies having a mandrel lumen and a wire retention fixture, the body of the second electrode set including a wire pass through channel, the wire retention fixtures and wire pass through channel opening onto the corresponding mandrel lumens, wherein the mandrel lumens, wire retention fixtures and wire pass through channel are monolithically formed in the corresponding bodies;
directly seating distal ends of first and second wire filers into the corresponding wire retention fixtures of the first and second electrode sets, respectively, such that the wire retention fixtures frictionally secure and retain the first and second wire filers;
arranging the first and second electrode sets in line with one another such that an intermediate portion of the first wire filer extends through the wire pass through channel in the second electrode set;
providing an outer tubing over the first and second electrode sets and wire filers extending from proximal ends of the first and second electrode sets; and
removing the linking portions of the first and second electrode sets to unlink the segmented electrodes.

2. The method of claim 1, further comprising bonding the first and second wire filers to the first and second electrode sets, respectively, wherein the seating operation retains the first and second filers directly engaged to, and seated in, the wire retention fixtures to form a non-hypo-tube connection there between during the bonding operation.

3. The method of claim 1, wherein the seating operation comprises introducing the distal ends of the first and second wire filers into the mandrel lumen and applying outward radial force to the distal ends of the first and second wire filers to snap and secure the distal ends into the corresponding wire retention fixtures.

4. The method of claim 1, further comprising: forming the mandrel lumen through the body of the first electrode set; forming at least two of the wire retention fixtures through the body of the first electrode set and opening onto the mandrel lumen; forming the wire pass through channel through the body of the first electrode set and opening onto the mandrel lumen; and forming an interstitial space through the body of the first electrode set and opening onto the wire pass through channel while leaving a linking portion of the body at a perimeter of the first electrode set to maintain first and second segmented electrodes in a linked state, the removing operation removing the linking portion to unlink the first and second segmented electrodes.

5. The method of claim 1, further comprising:
loading a non-metallic mandrel through the mandrel lumens in the first and second electrode sets, and through spacers separating the first and second electrode sets; and
applying a heat shrinking operation that melts the outer tube, mandrel and spacers to fill the mandrel lumens, and to fill voids and interstitial spaces within and surrounding the electrode sets and wire filers, the outer tube, spacers and forming a lumen-less body.

6. The method of claim 1, wherein the arranging operation further comprises loading a cord through the mandrel lumen of the second electrode set and thereafter, loading a proximal end of the first wire filer of the first electrode set through the wire pass-through channel in the second electrode set.

7. The method of claim 1, wherein, after the removing operation, the first and second electrode sets have an outer diameter of less than 25 mils when in an unlinked state with the linking portions removed.

8. The method of claim 1, wherein an inner diameter of the wire retention fixtures generally corresponds to an outer diameter of the corresponding first and second wire filers, the wire retention fixtures opening onto the corresponding mandrel lumens at transition zones that have a width that is less than an outer diameter of the corresponding first and second wire fliers.

9. A method for fabricating a neurostimulation stimulation lead comprising:
providing at least first and second electrode sets, each of which includes segmented electrodes joined with linking portions, the first and second electrode sets each including bodies having a mandrel lumen;
bonding distal ends of first and second wire filers to the first and second electrode sets, respectively;
arranging the first and second electrode sets separated by spacers and in line with one another such that an intermediate portion of the first wire filer extends through the second linked electrode set;
loading a non-metallic mandrel through the spacers and the mandrel lumens in the first and second electrode sets;
providing an outer tubing over the first and second electrode sets and over the wire filers extending from proximal ends of the first and second electrode sets;
applying a heat shrinking operation that melts the outer tube, mandrel and spacers to fill the mandrel lumens, voids and interstitial spaces within and surrounding the electrode sets and wire filers; and
removing the linking portions of the first and second electrode sets to unlink the segmented electrodes.

10. The method of claim 9, wherein the bodies of the first and second electrode sets each include a wire retention fixture, and the body of the second electrode set includes a wire pass through channel, the wire retention fixtures and wire pass through channel opening onto the corresponding mandrel lumens, wherein the mandrel lumens, wire retention fixtures and wire pass through channel are monolithically formed in the corresponding bodies.

11. The method of claim 10, further comprising bonding the first and second wire filers to the first and second electrode sets, respectively, wherein the seating operation retains the first and second filers directly engaged and seated in the wire retention fixtures to form a non-hypo-tube connection there between during the bonding operation.

12. The method of claim 10, further comprising directly seating distal ends of first and second wire filers into the corresponding wire retention fixtures of the first and second electrode sets, respectively, such that the wire retention fixtures frictionally secure and retain the first and second wire filers, wherein the seating operation comprises introducing the distal ends of the first and second wire filers into the mandrel lumen and applying outward radial force to the distal ends of the first and second wire filers to snap and secure the distal ends into the corresponding wire retention fixtures.

13. The method of claim 9, wherein the nonmetallic mandrel is formed of a polymer that melts to fuse with the outer tube and spacers to form a lumen-less body.

14. The method of claim 9, wherein, prior to the removing operation, the first and second electrode sets have an outer diameter of less than 40 mils when in a linked state with the linking portions joining the segmented electrodes, and wherein, after the removing operation, the first and second electrode sets have an outer diameter of less than 25 mils when in an unlinked state with the linking portions removed.

15. A neurostimulation stimulation lead comprising;
first and second electrode sets, each of which includes segmented electrodes, the first and second electrode sets including bodies having a mandrel lumen and a wire retention fixture, the body of the second electrode set including a wire pass through channel, the wire retention fixtures and wire pass through channel opening onto the corresponding mandrel lumens, wherein the mandrel lumens, wire retention fixtures and wire pass through channel are monolithically formed in the corresponding bodies;
wire filers having distal ends that are directly seating into the corresponding wire retention fixtures of the first and second electrode sets, respectively, such that the wire retention fixtures frictionally secure and retain the first and second wire filers;
the first and second electrode sets arranged in line with one another such that an intermediate portion of the first wire filer extends through the wire pass through channel in the second linked electrode set; and
an outer tubing provided over the first and second electrode sets and over wire filers extending from proximal ends of the first and second electrode sets.

16. The lead of claim 15, further comprising bonds joining the first and second wire filers to the first and second electrode sets, respectively, wherein the first and second filers are directly engaged and seated in the wire retention fixtures to form a non-hypo-tube connection there between during the bonding operation.

17. The lead of claim 15, wherein an inner diameter of the wire retention fixtures generally corresponds to an outer diameter of the corresponding first and second wire filers, the wire retention fixtures opening onto the corresponding mandrel lumens at transition zones that have a width that is less than an outer diameter of the corresponding first and second wire filers.

18. The lead of claim 15, further comprising a spacer between the first and second electrode sets and a non-metallic mandrel inserted through the spacers and the mandrel lumens in the first and second electrode sets, the non-metallic mandrel, spacers and outer tube in a melted state fused with one another to fill the mandrel lumens, voids and interstitial spaces within and surrounding the electrode sets and wire filers.

19. The lead of claim 15, wherein the first and second electrode sets have an outer diameter of less than 25 mils when in an unlinked state, the wire retention fixtures having an inner diameter that is substantially similar to an outer diameter of the wire filers, the wire pass through channels having an inner diameter larger than the outer diameter of the wire filers.

20. The lead of claim 15, wherein the first electrode set includes at least three segmented electrodes of approximately equal arcuate size, each of the at least three segmented electrodes having a corresponding wire retention fixture formed on an interior surface thereof at the mandrel lumen, the wire retention fixtures in a central region of the corresponding segmented electrodes as measured along an arcuate path of an exterior surface of the corresponding segmented electrode.
